(19) United States
(12) Patent Application Publication
(10) Pub. No.: US 2009/0155390 A1
(43) Pub. Date: Jun. 18, 2009

(54) Composition and method of treating temporary and permanent hearing loss

(76) Inventors: Josef M. Miller, Ann Arbor, MI (US); Colleen G. Le Prell, Gainesville, FL (US); D. Clark Bennett, Portage, MI (US); Peter A. Boxer, Dexter, MI (US)

Correspondence Address: HONIGMAN MILLER SCHWARTZ & COHN LLP, 444 WEST MICHIGAN AVENUE, KALAMAZOO, MI, 49007-3714, US

(21) Appl. No.: 12/313,527

(22) Filed: Nov. 21, 2008

Related U.S. Application Data
(63) Continuation-in-part of application No. 11/623,888, filed on Jan. 17, 2007.
(60) Provisional application No. 60/760,055, filed on Jan. 19, 2006.

(51) Int. Cl. A61K 33/00

(52) U.S. Cl. 424/722

(57) ABSTRACT

A composition for treating permanent and temporary hearing loss includes components that function through different biological mechanisms to provide an additive effect that is equal to or greater than a sum of the effect of the individual components. The composition includes a biologically effective amount of at least one scavenger of singlet oxygen, a donor antioxidant, a third antioxidant, and a vasodilator. A method of treating hearing loss includes the step of internally administering the composition including a biologically effective amount of the at least one scavenger of singlet oxygen, the donor antioxidant, the third antioxidant, and the vasodilator to a mammal within three days of trauma to a middle or inner ear of the mammal.

### RELATED APPLICATIONS

This patent application is a Continuation in Part Application to and claims priority to and all advantages of U.S. patent application Ser. No. 11/623,888, filed Jan. 17, 2007, which claims priority to Provisional Patent Application No. 60/760,055, which was filed on Jan. 19, 2006.

### GOVERNMENT LICENSE RIGHTS

The U.S. Government has a paid-up license in this invention and the right in limited circumstances to require the patent owner to license others on reason-able terms as provided for by the terms of grant number DC04058 awarded by the National Institutes of Health-National Institute of Deafness and Other Communication Disorders (NIH-NIDCD).

### BACKGROUND OF THE INVENTION

1. Field of the Invention

The present invention generally relates to a composition for treating hearing loss. More specifically, the present invention relates to a composition for treating temporary hearing loss that includes components that function through different biological mechanisms and provide an additive effect that is equal to or greater than a sum of the effects of the individual components, and a method of treating hearing loss including the step of administering the composition to a mammal prior to or within 3 days following trauma to the middle or inner ear.

2. Description of the Related Art

Extensive studies have been performed on compositions for treating hearing loss, along with methods of treating hearing loss using various compositions. In particular, antioxidants, among numerous other components, have been found to play a role in the prevention of noise-induced hearing loss. Specific antioxidants shown to be partially effective in reducing noise-induced hearing loss in animal models include glutathione (GSH)/glutathione monoethyl ester, N-acetylcysteine (NAC), resveratrol, allopurinol, R-phenylisopropyladenosine, and vitamins A, C, and E. Otoprotective effects of the above individual dietary antioxidants are well known in the art.

In addition to antioxidants, many other components have separately been investigated and found to be somewhat efficacious in treating hearing loss. Vasodilators are one class of components that have proven moderately useful for preventing hearing loss. It is known in the art that high levels of noise result in a decrease in blood flow to the inner ear, although the mechanism underlying this noise induced decrease has not been clear until very recently. On the basis of the observed decrease, it has long been speculated that this decrease in blood flow may lead to cell death in sensitive hair cells within a cochlea of the ear and accordingly an increase in blood flow may protect the inner ear cells from noise-induced death. Some vasodilators promote increased blood flow to the inner ear and, thus, help to protect the inner ear from trauma as a result of high levels of noise. Specific examples of vasodilators proven to partially prevent hearing loss include magnesium, betahistine, and hydroxyethyl starch (HES).

To date, little, if any, additive effects have been found to exist by combining many of the known components for treating hearing loss. Additive effects, as used herein, refer to effects that are equal to or greater than a sum of the effects of the individual components. For the most part, no greater effect is observed by combining many of the different components that are effective in treating hearing loss than the effect of the most effective individual component in the composition, i.e., combinations of agents are only as effective as the most effective single agent delivered alone. For example, FIG. 1 shows the results of experimentation relative to additive effects of betahistine and Trolox®. Trolox® is a water-soluble analogue of alpha-tocopherol (vitamin E). The experimental conditions are described in further detail in the Examples section below. As is evident from FIG. 1, the combined effect of Trolox® and betahistine in minimizing threshold shift, which correlates to hearing loss, is no greater than the effect of the most effective of either Trolox® or betahistine alone for any given experiment. As such, the combination of Trolox® and betahistine does not produce an additive effect in treating hearing loss.

As the understanding of the mechanisms by which the various antioxidants work to treat or prevent hearing loss has become clearer, it has been found that combinations of certain antioxidants that act via complementary, but different, biochemical mechanisms may be more effective than the individual antioxidants alone. However, given the volume and variety of components that are known to affect hearing loss, as well as lack of knowledge relative to specific mechanisms by which the components function, additive effects between components have not been recognized to date.

In spite of the fact that additive effects between various components have not been recognized to date, certain disclosures have been made that generally group together all known components for treating hearing loss. These disclosures do not teach with sufficient specificity combinations of specific components that exhibit additive effects in treating hearing loss. For example, U.S. Pat. No. 6,093,417 is directed to a composition to treat ear disorders. The composition is topically applied into an ear canal to treat the hearing disorder. Although the U.S. Pat. No. 6,093,417 patent is directed to a composition that may include many components that are known to be somewhat effective in reducing hearing loss alone, including vitamins A, C, and E along with vasodilators and magnesium salts, there is no recognition of an additive effect between any of the components. As is evident from the above description relative to FIG. 1, many combinations of components do not exhibit additive effects. As such, the random combination of agents disclosed in the U.S. Pat. No. 6,093,417 patent would not provide any greater effect for treating hearing loss if included in the composition. Furthermore, effectiveness of the individual components greatly varies between oral, intravenous, and topical administration, and compositions for treating hearing loss are formulated differently depending on the contemplated mode of administration. Finally, the U.S. Pat. No. 6,093,417 patent does not teach with sufficient specificity biologically effective amounts of each component that would be sufficient to produce an effect individually, let alone additive effects between the various components. Thus, the disclosure of the U.S. Pat. No. 6,093,417 patent provides no further teaching than what was already known about each of the components, i.e., that each component, when used individually, is modestly effective in preventing hearing loss.

Although many of the components that are used to treat or prevent hearing loss provide other beneficial functions and are included in multivitamins, known multivitamins do not include biologically effective amounts of the components sufficient to treat or prevent hearing loss. Furthermore, multivitamins are generally used as part of a regular dietary regimen and there is no data that suggests the use of multivitamins that include a specific combination and concentration of components to prevent hearing loss induced by noise or other stress.

The use of dietary antioxidants to prevent visual dysfunction is disclosed in U.S. Pat. No. 6,660,297. The U.S. Pat. No. 6,660,297 patent specifically discloses vitamins A, C, and E in amounts that may be biologically effective to treat or prevent hearing loss. However, a combination of vitamins A, C, and E alone is not sufficient to yield clinically significant protection against noise induced hearing loss and the addition of zinc and copper as disclosed in the U.S. Pat. No. 6,660,297 patent would not be expected to reduce hearing loss. Furthermore, there is no suggestion or teaching in the U.S. Pat. No. 6,660,297 patent to use the composition for anything other than treating visual dysfunction.

The use of various interdependent biofactors and biomolecules that are said to reduce the potential for cochlear hair cell death and nerve atrophy, and the hearing loss and possible deafness that accompany them, are disclosed in U.S. Pat. No. 6,524,619, to Pearson et al.

Thus, there is an opportunity to provide a composition and a method of treating hearing loss including the step of administering the composition that includes a specific combination of components having an additive effect that is equal to or greater than the sum of the effect of the individual components in treating hearing loss when used in biologically effective amounts.

### SUMMARY OF THE INVENTION AND ADVANTAGES

The subject invention provides a composition for treating hearing loss including components that function through different biological mechanisms to provide an additive effect that is equal to or greater than a sum of the effect of the individual components. The composition includes a biologically effective amount of at least one scavenger of singlet oxygen, a donor antioxidant, a third antioxidant, and a vasodilator. The composition comprises at least one scavenger of singlet oxygen, which is present for reducing free radicals that contribute to hearing loss. The donor antioxidant is present for reducing peroxyl radicals and inhibiting propagation of lipid peroxidation that also contributes to hearing loss. The vasodilator is present for preventing decreases in both cochlear blood flow and oxygenation that also contribute to hearing loss.

The subject invention also provides a method of treating hearing loss. The method includes the step of internally administering the composition of the subject invention including a biologically effective amount of at least one scavenger of singlet oxygen, the donor antioxidant, the third antioxidant, and the vasodilator to a mammal within three days of trauma to a middle or inner ear of the mammal.

The combination of at least one scavenger of singlet oxygen, the donor antioxidant, the third antioxidant, and the vasodilator, in the biologically effective amounts, provides an additive effect in treating hearing loss that is equal to or greater than a sum of the effect of the individual components. Even more, the composition of the subject invention should prove to be effective in treating hearing loss if administered as late as three days after trauma to the middle or inner ear of the mammal. As a result, the composition and method of treating hearing loss of the subject invention provide great promise in helping to minimize hearing loss resulting from trauma to middle or inner ears of mammals. Given the high incidence of noise-induced hearing loss in the general population worldwide, there is a great need for the composition and method of treating hearing loss of the subject invention in order to minimize socioeconomic effects that persist due to noise-induced hearing loss.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1 is a graph showing the effect of a Comparative Example of a composition used to treat hearing loss. It includes the combination of betahistidine and Trolox® (vitamin E analog) as a treatment for the reduction of a threshold shift in guinea pigs from baseline threshold sensitivity at 4, 8, and 16 kHz after exposure to 120 decibel SPL Octave Band Noise centered at 4 kHz for five hours;

FIG. 2 is a graph showing the effect of a composition of the subject invention including; Trolox, vitamins A and C, and magnesium, and comparative examples of compositions that include only some of those components, on average reduction of a threshold shift in guinea pigs from baseline threshold sensitivity at 4, 8, and 16 kHz after exposure to 120 decibel SPL Octave Band Noise centered at 4 kHz for five hours;

FIG. 3 is a graph showing the effect of the composition of the subject invention and Comparative Examples of compositions of FIG. 2 on an amount of missing hair cells in the region of the cochlea that is most damaged after the noise exposure specified above for FIG. 2; and

FIG. 4 is a graph showing the effect of the composition of the subject invention and Comparative Examples of compositions of FIG. 2 on an amount of missing hair cells in the whole cochlea after the noise exposure specified above for FIG. 2.

### DETAILED DESCRIPTION OF THE PREFERRED EMBODIMENT

A composition for treating hearing loss includes components that function through different biological mechanisms to provide an additive effect that is equal to or greater than a sum of the effect of the individual components. The composition is typically used for treating hearing loss resulting from trauma to a middle or inner ear of a mammal. The trauma may be further defined as mechanically-induced metabolic trauma, mechanical/metabolic trauma, stress trauma, stress-induced damage, or environmental stress. However, it is also possible that the composition may also be used to treat or prevent other types of hearing loss, including, for example, age-related hearing loss, antibiotic-induced hearing loss, and chemotherapeutic-induced hearing loss. The composition may further be used to prevent hearing loss during restoration surgery performed on the middle or inner ear.

The ear can be thought of as three parts working together to collect, amplify and transduce sound into neural impulses which are carried by the hearing nerve to the brain. The three parts of the ear are: the outer ear, the middle ear and the inner ear.

The outer ear consists of the part we see, the auricle and the ear canal. The opening in the auricle leads to the ear canal which is sealed at the eardrum, or tympanic membrane.

The middle ear is a region or space that contains several structures including the eardrum. The eardrum is a thin, flexible membrane that separates the outer ear from the middle ear. The middle ear is an air filled space that houses the three middle ear bones that transmit sound. The first bone is the hammer (malleus), which is connected to the anvil (incus), which is connected to the stirrup (stapes). These tiny bones are named to reflect their particular shapes. The middle ear is connected to the back of the nose (nasopharynx) by the eustachian tube. The stapes is coupled to the inner ear at an opening called the oval window. The eardrum and the three bones of the middle ear act to efficiently conduct sound vibrations in air (in the ear canal) to the fluid filled inner ear.

The inner ear is made up of both hearing (auditory) and balance (vestibular) components. The inner ear consists of a maze of fluid-filled tubes running through the temporal bone of the skull. There are three cavities in this space. The front portion is the snail-shaped cochlea, which functions in hearing. The cochlea is connected with the middle ear by two membrane-covered openings, the oval window (fenestra vestibuli) and the round window (fenestra cochleae). The oval window and round window open into the vestibule, at the base of the cochlea. The cochlea is a coiled tube around the modiolus that houses the hearing nerve. Reissner's membrane and the basilar membrane divide the cochlea longitudinally into three scalae. The scala vestibuli is on top, the scala tympani is on the bottom. The scala media is a triangular duct in the middle. The scala media is formed by Reissner's membrane, basilar membrane and the structure called the stria vascularis.

The fluid that fills the scala tympani and scala vestibuli is called perilymph; the fluid that fills scala media is called endolymph, each with specialized ionic and electrical properties. The organ of Corti rests on the basilar membrane within scala media. The sensory cells (hair cells) of this organ have tiny hairlike strands (cilia) that protrude into the fluid of the cochlea. Deflection of these hairs is converted to an electrochemical reaction in the hair cell which results in activation of the hearing nerve and the perception of sound.

The semicircular canals and the vestibule are the two parts of the inner ear involved with balance. The vestibule with sense organs that inform us of the position of the head in space is the central inner ear cavity that is bounded on one side by the oval window of the cochlea. The semicircular canals, attached to the vestibule, contain the sensory organs that tell us of the movement of the head through space.

It has been found that one result of noise trauma, or other stressors such as age and drugs as noted above, is that free radicals form in association with metabolic trauma. The free radicals then damage sensitive structures, such as hair cells, within the ear. Vasoconstriction also occurs as a result of the noise, which leads to decreased blood flow to the middle and inner ear and causes cell death that results in hearing loss. The underlying cause of vasoconstriction is noise-induced free radical formation. Specifically, one of the molecules formed in the inner ear as a result of the presence of free radicals is 8-isoprostane-2F alpha, which is a bioactive agent. The bioactive agent induces a constriction of blood vessels in the inner ear, which causes a reduction in blood flow.

While not being bound by any theory, it is believed that the composition of the invention reduces both permanent and temporary hearing loss, referred to as permanent threshold shift (PTS) and temporary threshold shift (TTS) respectively, because of its ability to counteract the free radical formation and the vasoconstriction that occurs with and may be the cause of hearing loss. The composition of the subject invention includes at least one scavenger of singlet oxygen, a donor antioxidant, a third antioxidant, and a vasodilator. Unexpectedly, it was found that compositions which include at least one scavenger of singlet oxygen, a donor antioxidant, a third antioxidant, and a vasodilator produce an effect that is greater than the effect of any one of those components alone, and is at least equal to or greater than a sum of the effects of each of the components.

Antioxidants act through a variety of mechanisms. The composition comprising at least one scavenger of singlet oxygen and the donor antioxidant are two different classes of antioxidants that act through different mechanisms. The third antioxidant, while typically a scavenger of singlet oxygen, may be a different antioxidant that acts through a different mechanism. Scavengers of singlet oxygen reduce free radicals that contribute to hearing loss. More specifically, by reducing free radicals, the scavengers of singlet oxygen prevent, among other damaging effects, the singlet oxygen from reacting with lipids to form lipid hydroperoxides. Lipid hydroperoxides play a role in causing hearing loss.

Even within the class of scavengers of singlet oxygen, it is believed that various antioxidants react at different sites within the body, and in particular, within cells to attenuate free radical formation. For example, one of the scavengers of singlet oxygen is typically vitamin A. Vitamin A is a generic term that captures a number of molecules with the biological activity of retinol or carotenoids. Primary dietary forms of vitamin A/retinol include retinol esters and beta-carotene. Beta-carotene is made up of a polyene chain of 11 conjugated double bonds with methyl branches spaced along the polyene chain, capped at both ends by cyclohexenyl rings with 1,1,5-trimethyl substitution. Other forms of vitamin A include xanxthophylls, astaxanthin, canthxanxin, lutein, and zeaxanthin, which include a backbone of beta-carotene with hydroxyl and/or carbonyl substitution on one or more of the cyclohexenyl rings. For purposes of the subject invention, vitamin A is typically present as beta-carotene. Beta-carotene is a powerful scavenger of singlet oxygen, as well as nitric oxide and peroxynitrite, and may also scavenge lipid peroxyl radicals within a lipophilic compartment of a mitochondrial membrane. Beta-carotene is an excellent scavenger of free radicals under normal physiological conditions present in most tissues.

In addition to vitamin A, other scavengers of singlet oxygen may also be present in the composition of the subject invention. For example, another scavenger of singlet oxygen that may be present is resveratrol. Resveratrol is more efficient at scavenging hydroxyl radicals than vitamin C, and the addition of resveratrol to the vitamins may have additive effects. The use of resveratrol in combination with other antioxidants (but not vitamins A, C, or E, and not the vasodilator magnesium or any other vasodilating substance), is known in the art to reduce age-related hearing loss.

The composition comprises at least one scavenger of singlet oxygen that is present in the composition in a biologically effective amount. For purposes of the subject invention, the biologically effective amount is further defined as an amount that is sufficient to produce an additive effect in a reduction in threshold shift when used in combination with other antioxidants and the magnesium. Additive effect, as used herein, refers to an effect that is equal to or greater than a sum of the effects of the individual components. In order to produce the additive effect, the composition comprises at least one scavenger of singlet oxygen that is typically present in the composition in a total amount of at least 830 international units (IU), alternatively from about 830 to 10,000 IU, or as another alternative from about 830 to 5900 IU for an adult dosage.

For example, in one embodiment, vitamin A is present in the amounts set forth above. As used herein, it takes about 0.3 micrograms (μ) of vitamin A to provide the equivalent of one IU of vitamin A. Thus, at least 830 international units (IU) is equivalent to at least 0.25 mg of vitamin A, from 830 to 10,000 IU of vitamin A is equivalent to about 0.25 to 3 mg of vitamin A, and from 830 to 5900 IU of vitamin A is equivalent to from about 0.25 to 1.8 mg vitamin A.

The retinol activity equivalents (RAE) for retinol (vitamin A) conversion to beta-carotene, a pro-vitamin A carotenoid, is about 1 mg of vitamin A is equivalent to 12 mg of beta carotene, making the conversion of IU to weight for beta-carotene about 3.6 micrograms (μ) of beta-carotene that are needed to provide the equivalent of about one IU of vitamin A. For the example above, in another embodiment where vitamin A is present as beta-carotene in the composition, a total amount of at least 3.0 mg of beta-carotene or at least 830 international units (IU) of vitamin A as beta-carotene, alternatively from about 3.0 to 36 mg or 830 to 10,000 IU vitamin A as beta-carotene, or as another alternative from about 3.0 to 21 mg or 830 to 5900 IU of vitamin A as beta-carotene is typically present for an adult dosage.

Specific amounts of the vitamin A present in the composition may be dependent on the body weight of the mammal. In one specific example, the amount of vitamin A present in the composition is about 0.021 mg/kg body weight. Thus, for an average human weighing about 70 kg, the amount of vitamin A present in the composition may be about 1.5 mg, or if the vitamin A is in the form of beta-carotene, the beta carotene may be present in an amount of about 18 mg.

It is to be appreciated that, when additional scavengers of singlet oxygen such as resveratrol are present in the composition in addition to vitamin A, the total amount of scavengers of singlet oxygen may be greater than the ranges set forth above for at least one scavenger of singlet oxygen, so long as at least one scavenger of singlet is present in the amounts set forth above. In addition, other scavengers of singlet oxygen may be used in place of vitamin A, so long as the amount of at the least one scavenger of singlet oxygen is present in the amount set forth above. When present, the resveratrol is typically included in the composition in an amount of at least 1 mg, more typically in an amount of from 10 mg to 1500 mg, most typically in an amount of from 15 mg to 1000 mg.

Whereas the composition comprises at least one scavenger of singlet oxygen prevents the initial formation of lipid peroxides, the donor antioxidant reduces peroxyl radicals and inhibits propagation of lipid peroxidation that contributes to hearing loss. More specifically, the donor antioxidant reacts with and reduces peroxyl radicals and thus serves a chain-breaking function to inhibit propagation of lipid peroxidation. As is evident from the chain-breaking function of the donor antioxidant in lipid peroxidation, the donor antioxidant functions within cell membranes. A specific donor antioxidant that is contemplated for use in the composition of the subject invention is vitamin E. Vitamin E is a generic term for all tocols and tocotrienol derivatives with a biological activity of alpha-tocopherol. Primary forms of vitamin E include vitamin E or alpha-tocopherol, salts of alpha-tocopherol like, but not limited to alpha-tocopherol acetate, and Trolox® (a water-soluble analogue of alpha-tocopherol commercially available from Hoffman-La Roche, Ltd. of Basel, Switzerland).

The donor antioxidant is typically present in the composition in an amount of at least 75 IU, alternatively from about 75 IU to 1500 IU, or as another alternative from about 150 IU to 800 IU. As known in the art, a conversion of IU to weight for vitamin E is 0.66 mg/IU. Thus, when the donor antioxidant is vitamin E, at least 75 IU of vitamin E is equivalent to at least 50 mg of vitamin E, from 75 to 1500 IU of vitamin E is equivalent to from 50 to 1000 mg of vitamin E, and from 150 to 800 IU of vitamin E is equivalent to from 100 to 550 mg of vitamin E. As with vitamins A and C, specific amounts of the vitamin E present in the composition may be dependent on the body weight of the mammal. In one specific example, the amount of vitamin E present in the composition is about 3.8 mg/kg body weight. Thus, for an average human weighing about 70 kg, the amount of vitamin E present in the composition may be about 267 mg.

In addition to at least one scavenger of singlet oxygen and the donor antioxidant, the composition further includes the third antioxidant. While the third antioxidant may be a scavenger of singlet oxygen, the third antioxidant may also be an antioxidant that functions through a different mechanism. When the third antioxidant is a scavenger of singlet oxygen, at least one scavenger of singlet oxygen is still present in the composition as a separate component from the third antioxidant, and is still present in the composition in the amounts set forth above for at least one scavenger of singlet oxygen. As a result of the third antioxidant being another scavenger of singlet oxygen, the resulting composition would have at least two scavengers of singlet oxygen.

The third antioxidant is typically vitamin C, which is a scavenger of singlet oxygen. It is to be appreciated that, although third antioxidant is typically vitamin C, other antioxidants may be used in place of the vitamin C, and the other antioxidants may function through different mechanisms than vitamin C. The term vitamin C applies to substances that possess antiscorbutic activity and includes two compounds and their salts: L-ascorbic acid (commonly called ascorbic acid) and L-dehydroascorbic acid. In addition to being known as ascorbic acid and L-ascorbic acid, vitamin C is also known as 2,3-didehydro-L-threo-hexano-1,4-lactone, 3-oxo-L-gulofuranolactone, L-threo-hex-2-enonic acid gamma-lactone, L-3-keto-threo-hexuronic acid lactone, L-xylo-ascorbic acid and antiscorbutic vitamin. Vitamin C is known to scavenge both reactive oxygen species and reactive nitrogen species. It can be oxidized by most reactive oxygen and nitrogen species, including superoxide, hydroxyl, peroxyl and nitroxide radicals, as well as such non-radical reactive species as singlet oxygen, peroxynitrite and hypochlorite. Vitamin C thus inhibits lipid peroxidation, oxidative DNA damage, and oxidative protein damage.

In contrast to vitamin A, which functions best under conditions present in most tissues, water-soluble vitamin C is an excellent free radical scavenger in an aqueous phase to thus reduce free radicals at a site different from that of vitamin A. More specifically, ascorbic acid functions to reduce free radicals in fluid, such as in cytoplasmic fluid and/or blood, before the free radicals reach cell membranes.

The third antioxidant is typically present in an amount of at least 4,000 IU, alternatively from about 4,000 to 40,000, or as another alternative from about 6,000 to 20,000 IU. Using vitamin C as an example for converting IU to weight units for the third antioxidant, as known in the art, a conversion of IU to weight for vitamin C is 0.05 mg/IU. Thus, at least 4,000 IU of vitamin C is equivalent to at least 200 mg of vitamin C, from 4,000 to 40,000 IU of vitamin C is equivalent to from 200 to 2,000 mg vitamin C, and from 6,000 to 20,000 IU vitamin C is equivalent to from 300 to 1,000 mg vitamin C. As with vitamin A, specific amounts of the vitamin C or other third antioxidant present in the composition may be dependent on the body weight of the mammal. In one specific example, the amount of vitamin C present in the composition is about 7.14 mg/kg body weight. Thus, for an average human weighing about 70 kg, the amount of vitamin C present in the composition may be about 500 mg.

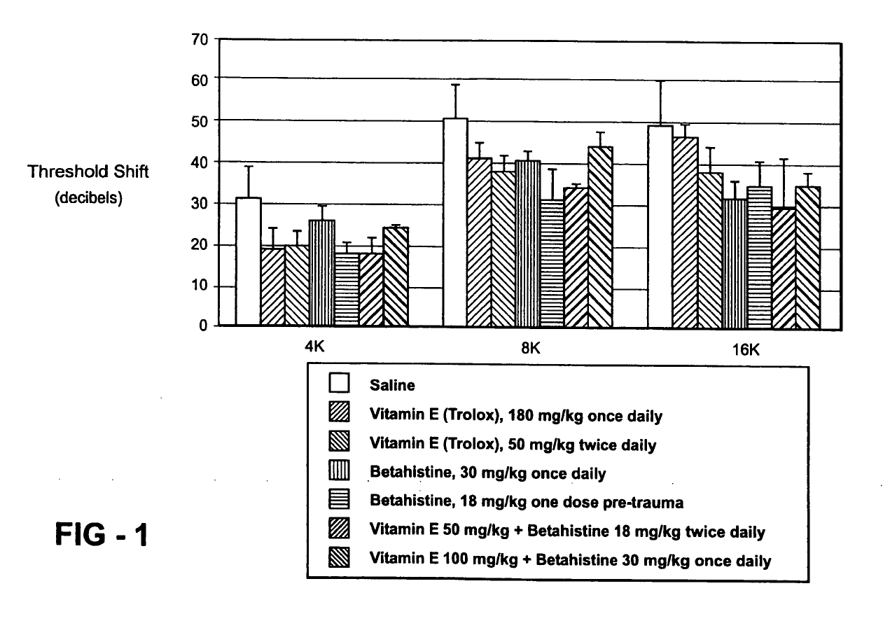

As set forth above, the composition further includes a vasodilator. Typically, the vasodilator includes magnesium; however, the vasodilator, for purposes of the subject invention, may include other vasodilators in place of or in addition to the magnesium, or may include only magnesium. Vasodilators are known in the art for use in preventing hearing loss. Vasodilators including magnesium prevent decreases in cochlear blood flow and oxygenation via biochemical mechanisms involving changes in calcium concentration and prostaglandins. Deficient cochlear blood flow and lack of oxygenation are factors that contribute to hearing loss by causing cell death in sensitive hair cells within a cochlea of the ear. Vasodilators including magnesium have also been found to improve the efficacy of immunosuppressant therapy or carbogen inhalation therapy in recovery from sudden hearing loss. Furthermore, it has been found that magnesium deficiency leads to increased calcium channel permeability and greater influx of calcium into cochlear hair cells, increased glutamate release, and auditory nerve excitotoxicity, each of which play a role in health of the middle and inner ear. Although the vasodilators are known in the art for treating hearing loss, the vasodilators, especially magnesium, exhibit an unexpected additive effect when combined with the biologically effective amounts of at least one scavenger of singlet oxygen, the donor antioxidant, and the third antioxidant, especially when at least one scavenger of singlet oxygen is vitamin A, the donor antioxidant is vitamin E, and the third antioxidant is vitamin C. The additive effect is greater than not only the most efficacious of the components for treating hearing loss, but typically greater than the sum of the effects of each of the components for treating hearing loss. While vasodilators other than magnesium are envisioned for purposes of the present invention, additive effects are not observed with all vasodilators. For example, betahistine, which is another known vasodilator, does not exhibit an additive effect as is evident from FIG. 1. Specific effects of the composition of the subject invention on treating hearing loss are described in further detail below.

The vasodilator including magnesium typically includes a magnesium salt or magnesium salt complex and, more specifically, magnesium sulfate, magnesium citrate or magnesium aspartate Other vasodilators including magnesium that may be suitable for purposes of the subject invention include; magnesium acetate, magnesium carbonate, magnesium chloride, magnesium fumarate, magnesium gluconate, magnesium glycinate, magnesium hydroxide, magnesium lactate, magnesium oxide, magnesium salicylate, and magnesium stearate. Other representative salts include but are not limited to; hydrobromide, hydrochloride, bisulfate, nitrate, arginate, ascorbate, oxalate, valerate, oleate, palmitate, laurate, borate, benzoate, phosphate, tosylate, maleate, fumarate, succinate, taurate, tartrate, naphthylate mesylate, glucoheptonate, lactobionate and laurylsulphonate salts.

Typically, the vasodilator is present in the composition in an amount of at least 50 mg. For example, when the vasodilator is magnesium, the magnesium is typically present in an amount of from about 50 to 450 mg, or alternatively from about 100 to 350 mg. As with vitamins A, C, and E, specific amounts of the vasodilator present in the composition may be dependent on the body weight of the mammal. In one specific example, the amount of the vasodilator including magnesium present in the composition is about 4.5 mg/kg body weight. Thus, for an average human weighing about 70 kg, the amount of the vasodilator including magnesium present in the composition may be about 315 mg.

Amounts of the typical components included in the composition, along with 1^{st} alternative and 2^{nd} alternative amounts, are summarized in Table 1 below.

TABLE 1

| | | | 1st Alternative | | 2^{nd} Alternative | |
|---|---|---|---|---|---|---|
| Component | Amount | | Amount | | Amount | |
| Vitamin A or | ≧830 | IU | 830-10,000 | IU | 830-5900 | IU |
| vitamin A as | | | | | | |
| beta-carotene | | | | | | |
| Vitamin C | ≧4,000 | IU | 4,000-40,000 | IU | 6,000-20,000 | IU |
| Vitamin E | ≧75 | IU | 75-1500 | IU | 150-800 | IU |
| Magnesium | ≧50 | mg | 50-450 | mg | 100-350 | mg |

Another use for this composition is as a combination containing vitamin A in the form of beta-carotene. The amount of beta-carotene is about 18 mg. Vitamin C is provided in the amount of about 500 mg. Vitamin E is provided in the amount of about 267 mg. And magnesium is provided in the amount of about 250 mg. In addition to the vitamins A, C, E and a vasodilator such as magnesium, other components may also be present in the composition for treating hearing loss. It is to be appreciated that the biologically effective amounts of the vitamins A, C, and E and magnesium may be lower within the above ranges for children than for the average human, based on lower US and European upper daily limits for children.

A method of treating hearing loss includes the step of internally administering the composition of the subject invention to a mammal. More specifically, the composition may be orally administered to the mammal, such as in the form of a tablet, capsule, liquid, gel, etc. Alternatively, the composition may be intravenously administered to the mammal through an IV or an injection of the composition. The composition may also be locally administered via the round window membrane of the cochlea. As a specific example, the vitamins A, C, and E, the vasodilator including magnesium, and any optional components may be first combined to form the composition, with the composition then administered to the mammal. Alternatively, the vitamins A, C, and E, the vasodilator including magnesium, and the other optional components may be separately administered, in which case the composition forms within the mammal.

There can be special advantages to producing the composition of the invention combined or mixed into a formulation that can then be administered as a daily therapeutic dose or daily TD. A therapeutic dose formulation will contain appropriate amounts the composition of the subject invention which includes at least one scavenger of singlet oxygen, a donor antioxidant, a third antioxidant, and a vasodilator; one example of which is the combination of the vitamins A, C, and E, with the vasodilator including magnesium, and any optional components desired. Once the components are mixed in the proper proportion, then it is possible to prepare one or more individual dosages that may contain an amount that is less than a full therapeutic dose. The daily TD is the amount of the composition of the invention used to treat one person for one day. It could be comprised of one or more dosages that are administered with the number of dosages varying depending on the size of the dose and the required daily TD. The daily TD can be comprised of one or more individual dosages that may contain an amount less than a daily TD, in which case several dosages would be administered to a person in order to provide the person with a daily TD. Dosages can be in the form of capsules or tablets and in this document capsules or tablets can include any type of pills, tablets, capsules, solid powders, liquids, suspensions, functional foods or any other orally ingestible formulation designed to be taken alone or after mixing with a suitable orally ingestible agent.

The composition of the invention can be produced as a formulation with the ingredients combined or mixed into a therapeutic dose (TD) suitable for administration in standard dosages in order to make up a daily TD. The administration of the composition then becomes especially easy and convenient. Here we describe how kits of such formulations could be produced and administered. In one example the doses that are provided are quite similar, they could even be nearly identical in size, color and shape For example, it is anticipated that the composition may be administered in capsules or tablets of size “O” (O), “OO” (double O), or “OOO” (triple O). These size capsules or tablets are commonly and conveniently used to administer vitamins and medications.

The range of the weight of the individual capsules or tablets can vary from 200 mg to 1500 mg, total weight including excipients, active and inactive ingredients. The range of the weights or amounts of the active ingredients of the individual capsules or tablets can vary as well with the following amounts provided as approximate amounts. Vitamin E from 25 to 100 mg/capsule or tablet. Vitamin C from 50 to 200 mg/capsule or tablet; Vitamin A from 1 to 8 mg/capsule or tablet in the form as beta-carotene. Magnesium from 25 to 125 mg/capsule or tablet. Further ranges and approximate amounts for these ingredients are provided in Table 2.

Table 2 shows the approximate weight of various size capsules and it describes approximately how much of each element would be in each capsule or approximate size shown. Amounts will vary according to capsule size and weight. Once produced, it would be a simple matter to quickly and conveniently provide the appropriate number of capsules to an individual in order to provide the correct daily TD. Approximate upper and lower ranges of active ingredients for the various capsule sizes are shown in Table 2 below.

TABLE 2

| Component | | Capsule Size | Capsule Size |
|---|---|---|---|
| active ingredient | Capsule Size O | OO | OOO |
| Total weight of | 500-570 | 700-800 | 900-1150 |
| capsule in mg* | | | |
| Vitamin E | 34.5-39.3 | 48.3-55.2 | 62.1-79.3 |
| mg active/capsule | | | |
| Vitamin C | 64.3-73.4 | 90.1-103 | 115.8-148 |
| mg active/capsule | | | |
| Vitamin A | 2.3-2.7 | 3.3-3.7 | 4.2-5.3 |
| mg active/capsule | | | |
| as Beta-carotene | | | |
| Magnesium | 40.7-46.4 | 57-65.1 | 73.2-93.6 |

*This is an approximate weight that includes optional components and inactive ingredients used to make the capsule.

The approximate number of dosages can be conveniently calculated, depending on the size of the capsule selected to administer the correct daily TD. As noted above, a daily therapeutic dose would comprise at least about the following amounts for a human.

In one specific example, the amount of vitamin A present in the composition is about 0.021 mg/kg body weight. Thus, for an average human weighing about 70 kg, the amount of vitamin A present in the composition may be about 1.5 mg, or if the vitamin A is in the form of beta-carotene, the beta carotene may be present in an amount of about 18 mg. According to Table 2 above, if one was using the composition formulated into size single O capsules (O) of a total capsule weight of about 500 mg, with Vitamin A as beta-carotene, one would administer at about 8 capsules (18/2.3=7.83) to an individual of about 70 kg. Similarly one would administer about 5 capsules (18/3.7=4.86) if formulated into a double O capsule (O) of a total capsule weight of about 800 mg. Of course the capsules or tablets could be scored, divided or broken and used as needed as part of the use of the composition as a kit.

Vitamin E. In one specific example, the amount of vitamin E present in the composition is about 3.8 mg/kg body weight. Thus, for an average human weighing about 70 kg, the amount of vitamin E present in the composition may be about 267 mg. According to Table 2 above, if one was using the composition formulated into size single O capsules (O) of a total capsule weight of about 500 mg, one would administer at least about 8 capsules (267/34.5=7.74) to an individual of about 70 kg. Similarly one would administer about 5 capsules or more (267/55.2=4.84) if formulated into a double O capsule (OO) of a total capsule weight of about 800 mg.

Using vitamin C as an example. In one specific example, the amount of vitamin C present in the composition is about 7.14 mg/kg body weight. Thus, for an average human weighing about 70 kg, the amount of vitamin C present in the composition may be about 500 mg. According to Table 2 above, if one was using the composition formulated into size single O capsules (O) of a total capsule weight of about 500 mg, one would administer at least about 8 capsules (500/64.3=7.77) to an individual of about 70 kg. Similarly one would administer about 5 capsules or more (500/103=4.83) if formulated into a double O capsule (OO) of a total capsule weight of about 800 mg.

Typically, the vasodilator is present in the composition in an amount of at least 50 mg. For example, when the vasodilator is magnesium, the magnesium is typically present in an amount of from 50 to 450 mg, most preferably from 100 to 350 mg. Thus, for an average human weighing about 70 kg, the amount of the vasodilator including magnesium present in the composition may be about 315 mg. According to Table 2 above, if one was using the composition formulated into size single O capsules (O) of a weight of about 500 mg, one would administer at least about 8 capsules (315/40.7=7.74) to an individual of about 70 kg. Similarly one would administer about 5 capsules or more (315/65.1=4.84) if formulated into a double O capsule (OO) of a weight of total capsule weight of about 800 mg.

Using calculations similar to that shown above one could quickly determine the minimum number of dosages required for different individuals in order to administer from a kit the daily therapeutic dose, or daily TD.

For purposes of the subject invention, hearing loss is objectively measured in terms of hearing threshold shifts. In guinea pig studies, permanent hearing loss and the efficacy of the composition of the subject invention for treating hearing loss is measured as an average difference in threshold shift from baseline threshold sensitivity at 4, 8, and 16 kHz, as compared to an untreated control, after exposure to 120 decibel SPL Octave Band Noise centered at 4 kHz for five hours. Larger differences in permanent threshold shift correlate to less hearing loss and greater efficacy of the composition for treating permanent hearing loss.

The relationship between Temporary Threshold Shift (TTS) and Permanent Threshold Shift (PTS) has not been clearly established. Some studies indicate that some mechanisms and structures may be common to both TTS and PTS. The many proposed histopathological correlates of TTS and PTS have been well reviewed. See, Saunders, J. C., Cohen, Y. E. and Szymko, Y. M. (1991). “The structural and functional consequences of acoustic injury in the cochlea and peripheral auditory system: a five year update,” J. Acoust. Soc. Am. 90, 136-146, and Nordmann, A. S., Bohne, B. A. and Harding, G. W. (2000). “Histopathological differences between temporary and permanent threshold shift,” Hear. Res. 139, 13-30. There is some evidence to suggest similar mechanisms underlie TTS and PTS. For example, at a genetic level, the Ahl allele in the C57 strain seemingly contributes to enhanced susceptibility to both TTS and PTS (as well as enhanced susceptibility to age-related hearing loss). See, Vazquez, A. E., Jimenez, A. M., Martin, G. K., Luebke, A. E. and Lonsbury-Martin, B. L. (2004). “Evaluating cochlear function and the effects of noise exposure in the B6.CAST+Ahl mouse with distortion product otoacoustic emissions,” Hear. Res. 194, 87-96. The results reported by Vazquez suggest the potential for a common genetic precursor that modulates TTS and PTS. Also linking TTS and PTS is the observation that both TTS and PTS can be reduced via “conditioning” exposures to moderate level noise prior to more intense noise, see Pukkila, M., Zhai, S., Virkkala, J., Pirvola, U. and Ylikoski, J. (1997). “The “toughening” phenomenon in rat's auditory organ,” Acta Otolaryngol. Suppl. (Stockh). 529, 59-62.

Consistent with the notion of similar mechanisms underlying TTS and PTS, it may be the case that TTS and PTS deficits represent a continuum of damage to hair cells and other elements within the organ of Corti. For example, in guinea pig ears that recovered from TTS, morphological damage was limited to the tips of stereocilia in the third row of outer hair cells (OHC) whereas ears from animals with PTS deficits had lesions of the inner hair cells (IHC) plus first row of OHC, or all three rows of OHC, with damage throughout the length of the stereocilia, Gao, W. Y., Ding, D. L., Zheng, X. Y., Ruan, F. M. and Liu, Y. J. (1992). “A comparison of changes in the stereocilia between temporary and permanent hearing losses in acoustic trauma,” Hear. Res. 62, 27-41. Stereocilia damage, with progressive damage to hair cells over time, has also been described. See Thorne, P. R., Gavin, J. B. and Herdson, P. B. (1984). “A quantitative study of the sequence of topographical changes in the organ of Corti following acoustic trauma,” Acta Otolaryngol. (Stockh). 97, 69-81, and Thorne, P. R., Duncan, C. E. and Gavin, J. B. (1986). “The pathogenesis of stereocilia abnormalities in acoustic trauma,” Hear. Res. 21, 41-49.

In contrast to the conclusions from the studies reported above, other results fail to support a relationship linking TTS and PTS. For example, there is a big difference between the molecular response of a TTS inducing sound when compared to a PTS inducing sound. Intense noise that induces PTS also causes the expression of transcription factors and cytokines. Moderate noise that induces TTS causes the down regulation of growth hormone. See, Cho, Y., Gong, T. W., Kanicki, A., Altschuler, R. A. and Lomax, M. I. (2004). “Noise overstimulation induces immediate early genes in the rat cochlea,” Brain Res. Mol. Brain. Res. 130, 134-148.

Following a TTS exposure Bcl-xl (anti-apoptotic) gene was expressed in outer hair cells, while PTS exposure led to Bak (pro-apoptotic) gene expression in the same cells, Yamashita, D., Minami, S., Ogawa, K. and Miller, J. M. (2005). “Bcl-2 genes regulate noise-induced hearing loss,” Abs. Assoc. Res. Otolaryngol. 28, 201.

In addition, the elegant use of survival-fixation techniques suggests PTS is best predicted by hair cell loss and neural degeneration, rather than TTS deficits Nordmann, A. S., Bohne, B. A. and Harding, G. W. (2000). “Histopathological differences between temporary and permanent threshold shift,” Hear. Res. 139, 13-30.

Given the uncertainty surrounding the relationship between TTS and PTS noted above, it is not surprising that the limited data available for studies of which antioxidant agents are generally effective against either or both TTS and PTS is also inconclusive and does not lead to any predictable conclusions about the relationship between the mechanisms of the two types of hearing deficits. For example, N-acetylcysteine (NAC), a glutathione precursor and reactive oxygen species (ROS) scavenger, has been effective in reducing PTS in noise-exposed guinea pigs, See Ohinata, Y., Miller, J. M. and Schacht, J. (2003). “Protection from noise-induced lipid peroxidation and hair cell loss in the cochlea,” Brain Res. 966, 265-273 and Duan, M., Qiu, J., Laurell, G., Olofsson, A., Counter, S. A. and Borg, E. (2004). “Dose and time-dependent protection of the antioxidant N-L-acetylcysteine against impulse noise trauma,” Hear. Res. 192, 1-9. NAC also seems to reduce PTS in chinchillas (delivered in combination with salicylate, see Kopke, R. D., Weisskopf, P. A., Boone, J. L., Jackson, R. L., Wester, D. C., Hoffer, M. E., Lambert, D. C., Charon, C. C., Ding, D. L. and McBride, D. (2000). “Reduction of noise-induced hearing loss using L-NAC and salicylate in the chinchilla,” Hear. Res. 149, 138-146.

However NAC appears to have little or no effect on TTS in humans or rodents. For studies in humans see Kramer, S., Dreisbach, L., Lockwood, J., Baldwin, K., Kopke, R. D., Scranton, S, and O'Leary, M. (2006). “Efficacy of the antioxidant N-acetylcystein (NAC) in protecting ears exposed to loud music,” J. Am. Acad. Audiol. 17, 265-278. For studies in rodents see, Kopke et al. 2000; Duan et al. 2004, both noted above.

Another agent and possible treatment of hearing loss that has been studied for its effects on both TTS and PTS, is D-methionine. D-Methionine appears to act by increasing endogenous GSH levels, by increasing bioavailable cysteine. Pre-noise treatment with D-methionine reduces PTS but not TTS, see Kopke, R. D., Coleman, J. K., Liu, J., Campbell, K. C. and Riffenburgh, R. H. (2002). “Candidate's thesis: enhancing intrinsic cochlear stress defenses to reduce noise-induced hearing loss,” Laryngoscope 112, 1515-1532.

Contrast the results noted above (NAC and D-methionine), with a study involving pre-treatment with ebselen. Ebselen is a glutathione-mimetic which reduces hydroperoxide formation. See Noguchi, N., Yoshida, Y., Kaneda, H., Yamamoto, Y. and Niki, E. (1992). “Action of ebselen as an antioxidant against lipid peroxidation,” Biochem. Pharmacol. 44, 39-44. Ebselen is reported to reduce both TTS and PTS. Treatments with ebselen and its effect in reducing both TTS and PTS have been reported in several articles. See Pourbakht, A. and Yamasoba, T. (2003). “Ebselen attenuates cochlear damage caused by acoustic trauma,” Hear. Res. 181, 100-108. Lynch, E. D., Gu, R., Pierce, C. and Kil, J. (2004). “Ebselen-mediated protection from single and repeated noise exposure in rat,” Laryngoscope 114, 333-337. Lynch, E. D. and Kil, J. (2005). “Compounds for the prevention and treatment of noise-induced hearing loss,” Drug Discov. Today 10, 1291-1298, and Yamasoba, T., Pourbakht, A., Sakamoto, T. and Suzuki, M. (2005). “Ebselen prevents noise-induced excitotoxicity and temporary threshold shift,” Neurosci. Lett. 380, 234-238.

As the studies cited above show, some antioxidants, like NAC or D-methionine, seem to be useful to treat PTS (guinea pigs and chinchillas) but not useful against TTS (humans and rodents). These two compounds are contrasted with ebselen which appears to treat both PTS and TTS. Given the difficulty of making good predictions about the efficacy of a drug's ability to treat both PTS and TTS it is surprising that the compounds of this invention are capable and have now been shown to treat both PTS and TTS.

Temporary threshold shift or TTS can be identified, studied and independently treated separate and apart from PTS, or permanent threshold shift, with the composition of this invention. A person suffering from TTS will have a partial to full recovery from hearing loss, unlike a victim of PTS. In many cases someone afflicted with TTS will have no permanent hearing loss. The definition of TTS, in this document, is a hearing loss that follows a traumatic event where at least some of the hearing loss is recovered within 7 days or less of the traumatic event. TTS typically results from a victim's exposure to less noise or trauma as compared to noise or trauma that causes PTS. Multiple afflictions or exposures leading to multiple episodes of TTS can lead to PTS. It is believed that those who are multiply afflicted with TTS in their youth are more likely to be afflicted with PFS in old age or experience an accelerated onset of age-related permanent hearing loss, as compared to mammals who are seldom afflicted with TTS.

TTS can be produced in mammals at any of 2, 4, 8, 16, 24 or 32 kHz, with at least a 10 decibel increase in the hearing threshold immediately or within 1 hour after exposure to a 90 decibel SPL sound, or more for 1 hour, or more. A symptom of TTS can also be produced in mammals at any of 2, 4, 8, 16, 24 or 32 kHz with as little as a 10 decibel shift, when compared to an untreated control, and it can occur immediately or within 1 hour or more after exposure to 110 decibel SPL Octave Band Noise centered at 4 kHz for four hours. TTS often resolves within 1, 2, or 3 days of the traumatic event. TTS frequently occurs as a result of trauma or noise exposure having less severity than the type of trauma or noise exposure that can result in PTS.

Reduction of a temporary hearing loss can result from a reduction in the magnitude of the initial TTS occurring immediately after noise exposure. It can also result from acceleration of the rate of recovery from a temporary hearing deficit; a shortening of the time period when TTS is greatest, within the first 1, 2, or 3 days after noise exposure. Accelerating the rate of TTS recovery, when TTS is greatest is beneficial because it is thought to decrease any permanent hearing loss that may also result from the instant noise exposure, and it can help minimize PTS resulting from the accumulated impact of multiple TTS incidents.

The compositions of this invention are particularly useful in the treatment of TTS. The compositions can provide a reduction in TTS of at least 9 decibels, at any of 2, 4, 8, 16, 24 or 32 kHz, when compared to untreated controls, within one hour after noise exposure to at least a 90 decibel SPL sound for 4 hours or more. As an alternative or in addition, the compositions of this invention can accelerate the rate of recovery from TTS by providing a decrease at any of 2, 4, 8, 16, 24 or 32 kHz, of at least 9 decibels in the TTS measured 1, 2, or 3 days after noise exposure to at least a 90 decibel SPL sound for 4 hours or more, when compared to the TTS measured at the same time in untreated controls.

The composition of the present invention is administered to the mammal within three days of trauma to a middle or inner ear of the mammal in order to alleviate threshold shift. It is to be appreciated that by administrating the composition within three days of trauma, treatment prior to trauma is also contemplated through the method of the present invention. Given the relationship between temporary threshold shift and permanent threshold shift, it is clinically beneficial to reduce temporary threshold shift. As such, the composition is preferably administered within one day of trauma to the middle or inner ear of the mammal. Even so, it is expected that treatment within three days with the composition of the present invention is substantially as effective in minimizing threshold shift as treatment within one day.

Treatment within three days is most appropriate when the mammal has sustained trauma to the middle or inner ear through unexpected loud noise or other trauma. Ideally, the composition is administered to the mammal prior to trauma to the middle or inner ear. Treatment prior to trauma is most feasible when the mammal is preparing for sustaining trauma to the middle or inner ear. For example, if a person will be firing a weapon or attending an event such as a rock concert, the person may begin treatment prior to sustaining the trauma to the middle or inner ear to attain the best results.

Another increasingly common use of the composition of the invention will be its administration prior to restoration surgery performed on the middle or inner ear. This is especially important as restoration surgery is often performed on individuals who still retain some hearing function. Each year thousands of people world wide with severe hearing loss undergo cochlear implant surgery. The success of this procedure in restoring hearing and significant speech comprehension has made it the ‘standard of care’ for deafness. Until recently this procedure was used only in patients with no marginal, remaining hearing. While the trauma from the cochlear implant surgery may cause some loss in the patient's residual hearing, this did not outweigh the benefits provided by the cochlear implant (CI). With the improvements in the technology of cochlear implants in the last few years, the success and speech perception benefit of the surgery has continued to increase. Now it is clear that for many patients, whom obtain minimal benefit from use of hearing aids, a CI may provide much greater benefit. With this expectation, the candidate population for CI has increased to include a population with greater residual hearing. In many of these patients, quite remarkable hearing benefits are being realized. It is clear that an important factor in the degree of benefit derived from a CI is the level of remaining hearing after surgery. Thus, the design and surgical approach used for implantation is being adapted to reduce surgical and implant induced trauma and increase the retention of residual hearing. While these changes have decreased the implant-linked reduction in residual hearing, this hearing reduction is still a significant factor in the patient population's outcome.

Cochlear implant surgery involves drilling on the otic capsule surrounding the cochlea (intense noise exposure), and insertion of the cochlear implant (mechanical trauma). These events stress the cells of the inner ear and the auditory nerve. The compositions of this invention, when taken as directed, will, in some cases, decreased implant-linked reduction from CI surgery of residual hearing.

After initial administration of the composition, the composition is typically administered to the mammal each day for at least five days following the trauma to the middle or inner ear of the mammal. Although excellent results have been achieved through such treatment, it is to be appreciated that other treatment regimens may also prove efficacious for purposes of the present invention.

The following examples, as presented herein, are intended to illustrate and not limit the invention.

### GENERAL INFORMATION FOR COMPARATIVE EXAMPLES 1 AND 2

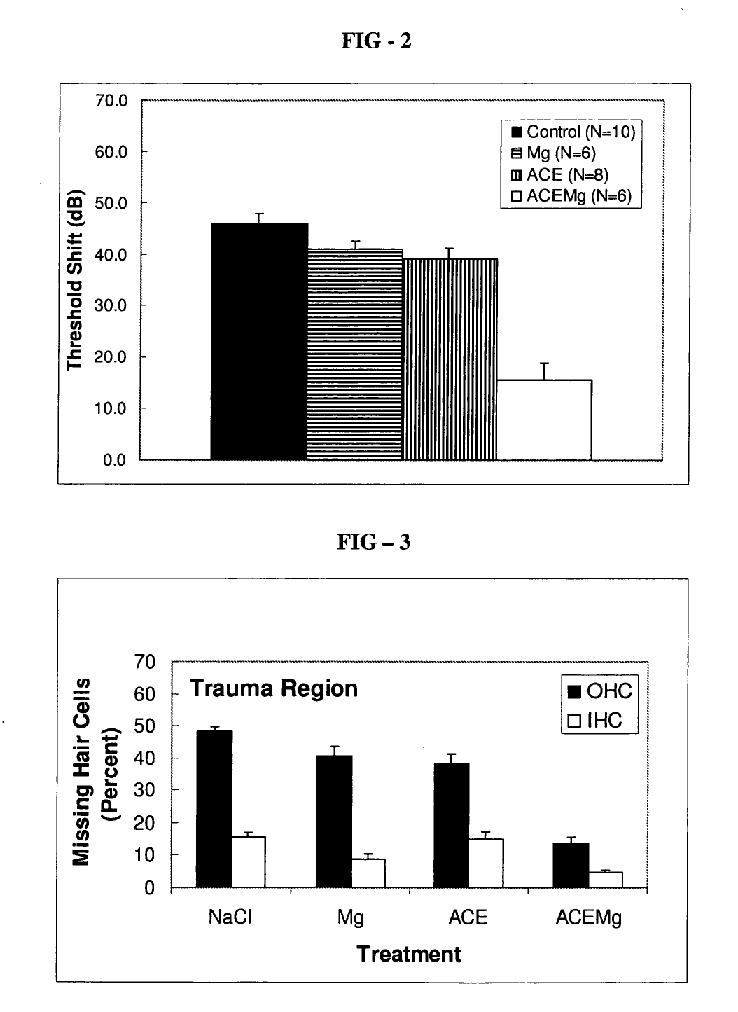

The method of treating hearing loss with the composition of the present invention is performed on guinea pigs (NIH outbred strain, 250-350 grams) due to their extensive use in auditory research, including studies on noise-induced hearing loss, and because they provide a model similar to humans in terms of development. Six guinea pigs are subject to treatment with the composition of the present invention. In order to determine efficacy of the composition of the present invention in treating hearing loss, baseline threshold sensitivity of the guinea pigs is measured binaurally using auditory brainstem response testing at 4, 8, and 16 kHz. The guinea pigs are then treated with vitamins A, C, E, and magnesium (referred to in FIG. 2 as “ACEMg”) in the amounts shown in Table 3. The amounts shown in Table 3 are 10 times expected human doses based on more rapid metabolism of guinea pigs relative to humans.

TABLE 3

| Component | Parts by Weight | |
|---|---|---|
| Vitamin A (beta-carotene) | 2.1 | mg/kg p.o. |
| Vitamin C (Ascorbic acid) | 71.4 | mg/kg s.c |
| Vitamin E (Trolox ®) | 26 | mg/kg s.c. |
| Magnesium (MgSO4) | 2.85 | mmol/kg s.c. |

One hour later, the guinea pigs are exposed to 120-dB SPL Octave Band Noise centered at 4 kHz for 5 hours to cause trauma to middle or inner ears of the guinea pigs. The noise is sufficient to cause permanent threshold shift, i.e., permanent hearing loss. The composition of the present invention is administered immediately post-exposure to the noise, and again each day for 5 days after the trauma. Ten days after the trauma, auditory sensitivity is measured using ABR. For ABR testing, the guinea pigs are anesthetized with 40 mg/kg ketamine and 10 mg/kg xylazine and placed on a warm heating pad in a sound attenuated chamber. ABR thresholds are determined at 4, 8 and 16 kHz frequencies. To test for the ABR thresholds, tone bursts 10 ms in duration (0.5 ms rise/fall) are presented at a rate of 17/sec. Up to 1024 responses are collected and averaged for each signal frequency to provide a measure of threshold shift at each frequency. Estimates of permanent hearing loss, shown in FIG. 2 in terms of threshold shift in decibels, are calculated as average threshold shift across ears and across frequencies. Similar results would be anticipated using other alternative measures of auditory or sensory cell function, such as psychophysical tests or otoacoustic emission measures.

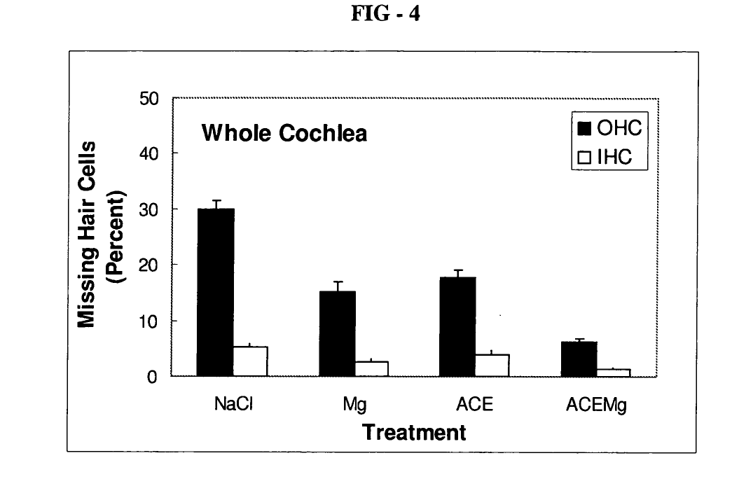

After ABR testing, the guinea pigs are deeply anesthetized and decapitated. Temporal bones are quickly removed, dissected open and fixed with 4% paraformaldehyde. The following day, an otic capsule, lateral wall, and tectorial membrane are removed, and a bony modiolus is carefully detached. Organ of Corti tissue, attached to the modiolus, is permeabilized with 0.3% Triton-X and incubated with rhodamine phalloidin diluted 1:100 in phosphate buffered saline (30 min). After washing the tissues, individual turns from the organ of Corti are dissected, mounted on microscope slides, and examined and photographed using a Leica DMRB epiflourescence microscope. Hair cell counts are conducted, and cytocochleograms are prepared as known in the art. Referring to FIGS. 3 and 4, percentages of missing inner hair cells (IHC) and outer hair cells (OHC) are determined based on the hair cell counts.

### Comparative Example 1

Guinea pigs are treated with other compositions in order to compare the efficacy of the composition of the present invention with the other compositions. For example, guinea pigs are separately treated in the same way as specified above in the Example with the following compositions: a saline (NaCl) composition as a control, a composition including only magnesium sulfate (2.85 mmol/kg) as the active ingredient, or a composition including only vitamins A (2.1 mg/kg beta-carotene), C (71.4 mg/kg ascorbic acid), and E (26 mg/kg Trolox®) (“ACE”) as the active ingredients. The guinea pigs are subjected to the same ABR testing, and the components of the ear are dissected, as described above in the Example to provide information on threshold shift and hair cell loss. Threshold shift and hair cell loss resulting from treatment with the other compositions are shown in FIGS. 2-4. After treatment according to the method of the present invention, outer hair cell loss in the whole cochlea is less than 10%, and inner hair cell loss in the whole cochlea is less than 5%. Outer cell hair loss in the trauma region is less than 20%, while inner hair cell loss in the trauma region is less than 10%.

### Comparative Example 2

Guinea pigs are treated with a composition of vitamin E alone, betahistine alone, or a combination of betahistine and vitamin E to determine if results similar to those achieved with the composition of the present invention including magnesium can be achieved by substituting betahistine for the magnesium. The results of treatment with vitamin E, betahistine, or combination of vitamin E and betahistine are shown in FIG. 1. In one comparative example, guinea pigs are treated once daily with 100 mg/kg vitamin E (Trolox®), 30 mg/kg Betahistine, or a combination of 100 mg/kg Trolox® and 30 mg/kg Betahistine. Five guinea pigs are treated with each of the different compositions. In another comparative example, four guinea pigs are separately treated with 50 mg/kg Trolox® twice daily, 18 mg/kg Betahistine (one dose immediately pre-noise exposure), or a combination of 50 mg/kg vitamin E (Trolox®) and 18 mg/kg Betahistine (N=4 animals per group). Control data are from 18 animals treated with saline delivered either IP (N=11) or IV (N=7). The guinea pigs are subjected to ABR testing, and the components of the ear are dissected, both as described above in the Example, to provide information on threshold shift and hair cell loss.

Referring to FIGS. 2-4, the effect in treating hearing loss with composition of the present invention including vitamins A, C, E, and magnesium is clearly greater than that associated with the effectiveness of a composition including only magnesium or only vitamins A, C, and E. More specifically, treatment with the composition of the present invention results in a threshold shift of less than 20 decibels, as averaged across all frequencies, while treatment with a control saline solution results in a threshold shift of about 45. As such, a difference in threshold shift between treatment with the composition of the present invention and treatment with the control of saline solution is over 25 decibels. Furthermore, the difference in threshold shift between the composition of the present invention and the control saline solution is greater than the sum of differences in threshold shifts for magnesium or vitamins A, C, and E alone and the control saline solution. Specifically, the sum of the differences in threshold shifts for magnesium and vitamins A, C, and E alone is about 12 decibels. Consistent with the reduction in noise-induced hearing loss, hair cell counts revealed significantly reduced sensory cell death with Mg or A, C, and E, with the greatest protection observed after treatment with vitamins A, C, E, and magnesium. More specifically, less than 10% of outer hair cells and less than 5% of inner hair cells in the whole cochlea are missing after treatment with the composition of the present invention including Mg and vitamins A, C, and E. In the trauma region, less than 20% of outer hair cells and less than 10% of inner hair cells in the whole cochlea are missing after treatment with the composition of the present invention including Mg and vitamins A, C, and E. As shown by FIGS. 3 and 4, the reduction in percentage of missing hair cells after treatment with the composition of the present invention is more than the sum of the reductions in missing hair cells observed when magnesium or vitamins A, C, and E are used alone as compared to the percentage of hair cell loss when saline is used.

Furthermore, as shown in FIG. 1, the Comparative Examples in which vitamin E, betahistine, or a combination of vitamin E and betahistine are used clearly do not exhibit the same additive effect that is observed when magnesium is used as the vasodilator in combination with the vitamins. More specifically, saline treated control animals (white bars) show the greatest hearing loss 10 days post noise. Animals treated with Vitamin E (Trolox: 100 mg/kg once daily or 50 mg/kg twice daily, diagonal lined bars) have less permanent hearing loss than control animals. Animals treated with the vasodilator betahistine (30 mg/kg, once daily, vertical striped bars; or one 18 mg/kg dose pre-noise, horizontal striped bars) show approximately the same amount of protection as those treated with vitamin E. Animals treated with a combination of vitamin E and betahistine (50 mg/kg Trolox twice daily+18 mg/kg betahistine twice daily, or 100 mg/kg Trolox once/daily+30 mg/kg betahistine once/daily; see dark bars with white diagonal hatch) do not have any additive protection beyond that of either single agent alone. As such, not all vasodilators are as effective as magnesium in combination with the vitamins.

### Comparative Example 3

Studies in animals have identified pathophysiological mechanisms of noise-induced hearing loss (NIHL), including free radicals formed in association with metabolic stress and reduced blood flow. Described is a therapeutic intervention with vitamins C, E, A, and magnesium that will reduce temporary noise-induced hearing deficits. Guinea pigs with normal auditory brainstem response (ABR) thresholds were implanted with a round window electrode used for compound action potential (CAP) threshold tests and normal hearing after implantation was verified. One week later, the animals were exposed to octave band noise (centered at 4-kHz) at 110-dB SPL for 4 hours. CAP thresholds were evaluated 1 day pre-noise, as well as 1 hour, 1 day, 3 days, 5 days, and 7 days post-noise. Both temporary threshold shift (TTS) and permanent threshold shift (PTS) deficits were measured. Two-thirds of the animals were treated with a micronutrient dose disclosed here that was shown to effectively reduce PTS (N=20, with 10 animals treated QD and 10 animals treated BID), while the remainder were saline-treated controls (N=10). Hair cell counts were used to confirm the lack of sensory cell death in the cochlea. A decrease in the intensity of the TTS and an increase in the rate of the early recovery of the TTS deficit were demonstrated, along with a reduction in PTS.

A total of 31 pigmented male guinea pigs (250-300 g; Elm Hill Breeding Labs, Chelmsford, Mass.) were used. Male guinea pigs were selected based on description of sex differences in ROS detoxification (Julicher et al., 1984), activity of glutathione S-transferase in the cochlea (El Barbary et al., 1993), and susceptibility to NIHL (McFadden et al., 1999). All subjects underwent a single surgical intervention approximately 1-week after arrival at the University of Michigan. The experimental protocol was reviewed and approved by the University Committee for the Care and Use of Animals (UCUCA), and all procedures conformed to the National Institutes of Health Guidelines for the Care and Use of Laboratory Animals.

Surgical Procedure. The surgical procedure was closely modeled after those we have described previously (Le Prell et al., 2004; 2005). Animals were anesthetized (40 mg/kg ketamine, 10 mg/kg xylazine), the bulla was exposed and gently opened using a post-auricular approach, and then a sterile ball electrode (0.25 mm diameter, constructed of teflon-coated platinum-iridium wire) was carefully placed on the round window membrane. A ground wire was inserted into the middle ear via the defect in the bulla, and carboxylate cement (Durelon, ESPE, Germany) was used to seal the bulla defect and permanently fix both the cannula and the electrodes in place. The opposing ends of the electrodes, soldered to a two-pin connector (HSS-132-G2, Samtec Inc., IN) prior to the onset of the surgical procedure, were fixed to the skull using methyl methacrylate cement (Jet Repair Acrylic, Lang Dental Manufacturing, IL). The post-auricular incision was then sutured and the incision cleaned. The indwelling electrode was used during subsequent sound-evoked electrophysiological testing.

Electrophysiological testing. The sound-evoked whole nerve compound action potential (CAP) was measured 1 day prior to and immediately, 1 day, 3 days, 5 days, and 7 days subsequent to noise exposure. Animals were anesthetized (29 mg/kg ketamine, 1.2 mg/kg xylazine, 0.6 mg/kg acepromazine), and placed on a warm heating pad to maintain body temperature during CAP tests. Acoustic stimuli were brief pure-tone stimuli (2, 4, 8, 16, 24, and 32 kHz) presented at levels ranging from 5 to 90-dB SPL in 5-10 dB increments (5, 10, 15, 20, 25, 30, 40, 50, 60, 70, 80, and 90 dB SPL). Acoustic signals were 5-msec in duration, with a 0.5-msec rise-fall time; signals were presented at a rate of 10/sec with 128 repetitions per frequency/level combination. Acoustic stimuli were generated using Tucker-Davis Technology (TDT; Alachua, Fla.) System III hardware and SigGenRP software. Signals were converted to analog, attenuated to set level (PA5), and presented using an ECI transducer coupled to the animals' ear canal via vinyl tubing. Cochlear potentials were digitally filtered (300-3000 Hz) using BioSigRP (TDT). CAP threshold was defined as the sound level that produced a 75-μV response; threshold estimates were determined using linear interpolation.

Six animals (AUR 2, 6, 11, 18, 20, and 29) were excluded from further analysis as threshold sensitivity measured during baseline testing revealed thresholds that were elevated by 2-3 standard deviations relative to the mean. To verify that there were no systematic differences in pre-treatment sensitivity across groups, baseline threshold data were compared via ANOVA, using the Greenhouse-Geisser correction for sphericity, after excluding animals with grossly elevated thresholds. Frequency (2, 4, 8, 16, 24 and 32 kHz) was treated as a within-subject factor and treatment (saline, QD micronutrients, and BID micronutrients) was the between-subject factor. Baseline CAP thresholds varied with frequency (F=31.356; df=1.5, 33.5; p<0.001) but did not vary as a function of group (F=1.403; df=2.22; p=0.267), and there was no statistically reliable interactions for frequency×group (F=1.414; df=3.33.5; p=0.256).

Several “single-day” post-noise data sets were excluded from analysis; these were data sets in which sound-evoked electrophysiological “responses” were indistinguishable from the noise floor, across levels and across frequencies, despite robust sound-evoked response during the preceding and the subsequent test sessions. The lack of response was neither noise-induced nor biological in origin. Noise-induced deficits are clearly frequency specific, and biological pathology that results in sudden, profound sensorineural deficit (i.e., lack of any detectable neural sound-evoked response across frequencies at 90 dB SPL), would not be fully reversible over 24-48 hour intervals. Animals with these transient response issues were distributed across groups; a total of 3-5 data sets were eliminated on days 1, 3, and 7. None of the day 5 data sets were excluded.

Implanted animals were assigned to one of three groups: saline-treated control (1 cc, s.c.), or micronutrients as in our previous investigation (Le Prell et al., 2007). All treatments were initiated 1 day prior to noise exposure, prior to CAP testing, and continued daily at 24-hour intervals until day 5 post-noise, for a total of 6 daily treatments. Total daily doses were as follows: vitamin A, 2.1 mg/kg beta-carotene delivered orally (p.o.); vitamin C, 71.4 mg/kg ascorbic acid (s.c.); vitamin E, 26 mg/kg (s.c.) with vitamin E delivered in the form of Trolox, a cell-permeable, water-soluble derivative of vitamin E; and magnesium, 2.85 mmol/kg magnesium sulfate (s.c.). The QD experimental group received the entire daily dose during one daily treatment. The BID experimental group received two equal daily treatments, each providing ½ of the total daily dose of the active agents. These treatments were separated by approximately 8 hours to maintain more constant serum levels of the active agents. All test substances were purchased from Sigma-Aldrich (St. Louis, Mo.) (beta-carotene, #C9750, CAS 7235-40-7; L-threoascorbic acid, #A5960, CAS 50-81-7; Trolox, Fluka Chemika #56510, CAS 53188-07-1; magnesium sulfate, #M7506, CAS 7487-88-9). Manipulating the dose delivery paradigm did not influence the efficacy of the active agents.

All subjects were exposed to octave-band noise (centered at 4 kHz, 110 dB SPL, 4 hours). This noise exposure is shorter, and less intense than, the 5-hour 120-dB SPL exposure previously used to induce permanent hearing deficits (Yamashita et al., 2004; Yamashita et al., 2005); the combination of micronutrients effectively reduces permanent hearing loss associated with that louder, longer, exposure (Le Prell et al., 2007). Other conditions of the exposure were as in our previous investigations. Specifically, animals were exposed, two at a time in separate cages, in a ventilated sound exposure chamber fitted with speakers (Model 2450H, JBL, Salt Lake City, Utah) driven by a noise generator (ME 60 graphic equalizer, Rane, Mukilteo, Wash.) and power amplifier (HCA-1000 high current power amplifier, Parasound Products, San Francisco, Calif.). Sound levels were calibrated (Type 2203 precision sound level meter, Type 4134 microphone, Bruel and Kjar Instruments, Norcross, Ga.) at multiple locations within the sound chamber to ensure uniformity of the stimulus, using a fast Fourier transform network analyzer with a linear scale. The stimulus intensity varied by a maximum of 3 dB across measured sites within the exposure chamber. During noise exposure, noise levels were monitored using a sound level meter, a pre-amplifier, and a condenser microphone positioned in the center of the chamber at the level of the animal's head. All days are relative to noise exposure, where Day 0 is the day of the noise exposure: Implant electrode on round window membrane, left ear only.

| | |
|---|---|
| 1 Day Pre-Noise: | Baseline CAP left ear only (compound action |
| | potential) |
| | Start treatment: |
| | Grp 1: Saline control - AM treatment only |
| | Grp 2: Auraquell ™ - AM treatment only |
| | Grp 3: Auraquell ™ - AM & PM Treatment |
| | (~8 hrs. apart) |
| Day 0: | AM treatments will be 1 hour Pre-Noise - all Grps. |
| | Noise-expose all groups |
| | 1 hour Post-Noise:CAP |
| | 3 hours Post-Noise PM treatments (Grp 3) |
| Day 1 Post-Noise: | AM treatments - all Grps. (similar time each day) |
| | CAP |
| | PM treatments (similar time each day) |
| Day 2 Post-Noise: | AM treatments - all Grps. (similar time each day) |
| | PM treatments - Grp 3 (similar time each day) |
| Day 3 Post-Noise: | AM treatments - all Grps. (similar time each day) |
| | CAP |
| | PM treatments (similar time each day) |
| Day 4 Post-Noise: | AM treatments - all Grps. (similar time each day) |
| | PM treatments - Grp 3 (similar time each day) |
| Day 5 Post-Noise: | AM treatments - all Grps. (similar time each day) |
| | CAP |
| | PM treatments (similar time each day) |
| Day 6 Post-Noise: | AM treatments - all Grps. (similar time each day) |
| | PM treatments - Grp 3 (similar time each day) |
| Day 7 Post-Noise: | CAP |

Auraquell ™ is a combination of Vit. A, C, E, and Mg salt as described in paragraph 0097 above.

### Euthanasia, Harvest Cochleae

On day 7, after CAP measurement, the deeply anesthetized animals were decapitated and the cochleae were immediately removed for immunohistochemical staining with rhodamine phalloidin and hair cell counts. Upon removal, cochleas were transferred into 4% paraformaldehyde in 0.1M phosphate-buffered saline (PBS, pH 7.4). Under a dissecting microscope, the bone nearest the apex and the round and oval windows was opened, followed by gentle local perfusion from the apex. The tissue was kept in fixative for 12 hours, then the bony capsule and the lateral wall tissues were removed, and the modiolar core was carefully removed from the temporal bone. Following permeabilization with Triton X-100 (0.3%, 30 min), the organ of Corti was stained for f-actin using rhodamine phalloidin (1%, 60-120 min) to outline hair cells and their stereocilia (Raphael and Altschuler, 1991). After washing the tissues with PBS, the organ of Corti was dissected and surface preparations were mounted on glass slides. The tissues were observed under fluorescence microscopy, and the number of missing inner hair cells and outer hair cells were counted from the apex to the base in 0.19 mm segments (as described in Yamashita et al., 2004). Counting was begun approximately 0.76-1.14 mm from the apex, thus omitting the initial irregular most-apical part of the cochlear spiral. Percentages of hair cell loss in each 0.19 mm length of tissue were plotted along the cochlear length.

Statistical comparisons were performed using SPSS for Windows (version 15.0). Statistical reliability of group differences in threshold and threshold shift were examined for each time point via ANOVA; frequency (2, 4, 8, 16, 24 and 32 kHz) was treated as a within-subject factor and treatment (saline, QD micronutrients, and BID micronutrients) was the between-subject factor. Adjustment for multiple comparisons was accomplished using the Bonferroni correction. Pair-wise comparisons during initial analyses revealed that there were no reliable differences between SID and BID micronutrient groups on any of the functional measures, thus, all treated subjects were combined into a single group for subsequent analyses.

The Results showed that while subjects in both treated groups showed compelling deficits in threshold sensitivity subsequent to noise exposure, there were some subtle differences in the time course of recovery. Recovery was largely complete in the control animals by day 3 and recovery continued to at least day 5 in the treated animals. When normalized to baseline, noise-induced threshold deficits tended to be smaller in animals treated with a combination of antioxidant agents and magnesium. The most robust differences were observed at the higher test frequencies. There was a trend toward threshold differences immediately post-exposure, with the 8 kHz test frequency showing the greatest difference. Both saline-treated control animals and micronutrient-treated animals experienced the greatest temporary threshold shift (TTS) at 8 kHz, with the amount of threshold shift being approximately 10 dB smaller in treated animals compared to controls (p=0.054). Differences between treated and untreated groups were more apparent on 1 day post-noise. Treated subjects had significantly less threshold shift at 24 and 32 kHz, approximately 20 and 15 dB respectively, compared to control subjects (p's<0.05). The difference at 8 kHz was approximately 15 dB, but was less significant (p=0.101), with no evidence of group differences at other test frequencies. Thus, functional protection was largely at higher frequencies, corresponding to more basal regions of the cochlea. Both treated and control groups showed significant recovery of TTS deficits on days 3 and 5, with no statistically reliable group differences in threshold shift at any frequency (all p's>0.05). Threshold differences between groups re-emerged at the last (7-day post-noise) time point. The 7-day, post-noise, time point showed that the reduction in threshold shift at 24 kHz was about 10 dB (p<0.05) for the treated group as compared to the untreated saline controls. Protection at this latter time point, when threshold deficits are largely permanent (see, for example, Yamashita et al., 2004), is consistent with previous reports that micronutrient treatment can reduce PTS (Le Prell et al., 2007). Corresponding to the small threshold deficits measured at 7 days post-noise, there was little hair cell loss in either subject group. Noise-induced cell death was limited to a small lesion of row 1 OHCs approximately 10-12 mm from the apex, and statistical comparisons failed to reveal any significant group differences.

The significant reduction in noise-induced hearing loss in animals treated with both dietary antioxidants and magnesium was accompanied by an increase in the amplitude of the sound-evoked neural response at supra-threshold levels, shows preservation of neural function contributed to the reduction in noise-induced threshold shifts.

In Summary. Subjects in the treated group had smaller threshold shifts than did control animals, immediately post-exposure as well as 24 hours after noise exposure. In addition, neural response amplitude was consistently greater in treated animals. This data demonstrates the treatment's ability to; reduce the level of temporary hearing loss, and accelerate the rate of recovery from TTS.

All subjects in all groups showed significant recovery from TTS, seven days after noise exposure. The subjects treated with the micronutrients had the best hearing outcomes at this final test time, indicating that the treatment also reduced the minor permanent hearing loss that occurred because of the noise exposure.

The invention has been described in an illustrative manner, and it is to be understood that the terminology which has been used is intended to be in the nature of words of description rather than of limitation. Obviously, many modifications and variations of the present invention are possible in light of the above teachings, and the invention may be practiced otherwise than as specifically described.

What is claimed is:

1. A method of treating, preventing or reducing the severity or duration of temporary hearing loss, experienced by a mammal, comprises administering the following composition to said mammal:
a) at least one scavenger of singlet oxygen, selected from any of:
i) vitamin A, wherein said vitamin A scavenger of singlet oxygen has the vitamin A equivalence of at least 830 IU,
ii) beta-carotene, wherein said beta-carotene scavenger of singlet oxygen has an activity from about 830 to 5,900 IU, (3 to 21 mg) or
iii) resveratrol, wherein this scavenger of singlet oxygen has a singlet oxygen scavenging activity equivalent to at least 830 IU of vitamin A,
or any combination thereof where the total activity of said scavenger of singlet oxygen has at least the equivalent of 830 IU of vitamin A;
b) a donor antioxidant which is vitamin E and is present in an amount of at least 75 IU;
c) a third antioxidant which is vitamin C and is present in an amount of at least 4,000 IU and;
d) a vasodilator which is magnesium and it is present in an amount of at least 50 mg;
wherein said composition is administered within 3 days prior to trauma to the middle or inner ear or within three days after trauma to the middle or inner ear of a mammal.

2. The method of claim 1 wherein said composition is administered within 3 days prior to trauma to the middle or inner ear of said mammal.

3. A method of claim 1 wherein said composition is administered within three days after trauma to the middle or inner ear of the mammal.

4. A method of claim 1 wherein the composition is administered within one day before, on the day of, or within one day after, trauma to the middle or inner ear of said mammal.

5. A method of claim 1 further comprising the step of internally administering the composition to the mammal each day for at least five days following the trauma to the middle or inner ear of the mammal.

6. The method of claim 2 wherein the trauma is caused by surgery to the middle or inner ear.

7. The method of claim 6 wherein the surgery is cochlear implant (CI) surgery.

8. A method of claim 1 wherein the amount of said hearing loss experienced by said mammal is an increase in temporary threshold shift (TTS) from a threshold sensitivity at any of 2, 4, 8, 16, 24 or 32 kHz, of at least 10 decibels, immediately or within 1 hour of the exposure, to 90 decibel or more of SPL sound for one hour or more.

9. A method of claim 8, wherein the amount of said hearing loss that the mammal looses, at any of 2, 4, 8, 16, 24 or 32 kHz, is reduced by at least 9 decibels, when compared to untreated controls, when such loss is measured immediately or within 1 hour of the sound exposure.

10. A method of claim 8, wherein the amount of said hearing loss recovery as a result of treatment from the composition is demonstrated by an increased rate of decline in the TTS of a treated mammal as compared to the rate of decline in the TTS of an untreated control mammal, when comparing similar times, 1, 2, or 3 days after sound exposure and the treated mammal shows a greater decrease of at least 9 decibels over the untreated mammal at any of 2, 4, 8, 16, 24 or 32 kHz.

11. A method of claim 1 wherein said scavenger of singlet oxygen is beta-carotene.

12. A method of claim 1 wherein the composition is administered from a kit, wherein said kit is comprised of:
a) one or more oral dosages and a label,
wherein said label describes the contents of the kit and directions for administering a daily therapeutic dose (TD);
b) wherein said kit is capable of dispensing one or more daily TDs;
c) wherein said daily TDs are similar in size and shape;
d) wherein said daily TDs are comprised of capsules or tablets that range in size from capsule size 0 to 000 and total weights of about 200 to 1500 mg per capsule with a per capsule or tablet amount of the following range for the following ingredients:
i) vitamin E from 25 to 100 mg/capsule or tablet;
ii) vitamin C from 50 to 200 mg/capsule or tablet;
iii) vitamin A from 1 to 8 mg/capsule or tablet in the form as beta-carotene, and
iiii) magnesium from 25 to 125 mg/capsule or tablet.

13. A method of claim 14 wherein the composition is administered from a kit, wherein said kit is comprised of capsules or tablets forming a daily dose wherein said daily dose comprises a combination of all of the active ingredients shown in any column of Table 2a herein, wherein the total weight of the capsule in mg is an approximate weight that includes optional components and inactive ingredients used to make the capsule;
TABLE 2a

| Component | | Capsule Size | Capsule |
|---|---|---|---|
| active ingredient | Capsule Size O | OO | Size OOO |
| Total weight of | 500-570 | 700-800 | 900-1150 |
| capsule in mg | | | |
| Vitamin E | 34.5-39.3 | 48.3-55.2 | 62.1-79.3 |
| mg active/capsule | | | |
| Vitamin C | 64.3-73.4 | 90.1-103 | 115.8-148 |
| mg active/capsule | | | |
| Vitamin A | 2.3-2.7 | 3.3-3.7 | 4.2-5.3 |
| mg active/capsule | | | |
| as Beta-carotene | | | |
| Magnesium | 40.7-46.4 | 57-65.1 | 73.2-93.6 |

.(period)

14. A composition for treating hearing loss experienced by a mammal wherein said composition comprises:
a) at least one scavenger of singlet oxygen, selected from any of:
i) vitamin A, wherein said vitamin A scavenger of singlet oxygen has the vitamin A equivalence of at least 830 IU,
ii) beta-carotene, wherein said beta-carotene scavenger of singlet oxygen has an activity from about 830 to 5,900 IU, (3 to 21 mg) or
iii) resveratrol, wherein this scavenger of singlet oxygen has a singlet oxygen scavenging activity equivalent to at least 10,000 IU of vitamin A,
or any combination thereof where the total activity of said scavenger of singlet oxygen has at least the equivalence of 830 IU of vitamin A;
b) a donor antioxidant which is vitamin E and is present in an amount of at least 75 IU;
c) a third antioxidant which is vitamin C and is present in an amount of at least 4,000 IU and;
d) a vasodilator which is magnesium and it is present in an amount of at least 50 mg;

15. A composition of claim 14 wherein the amount of said hearing loss is an increase in temporary threshold shift (TTS) in a mammal from a threshold sensitivity at any of 2, 4, 8, 16, 24 or 32 kHz, of at least 10 decibels, immediately or within 1 hour of the exposure, to 90 decibel or more of SPL sound for one hour or more.

16. A composition of claim 14, wherein the amount of said hearing loss that the mammal looses at any of 2, 4, 8, 16, 24 or 32 kHz, is reduced by at least 9 decibels, when compared to untreated controls, when such loss is measured immediately or within 1 hour of the sound exposure.

17. A composition of claim 14, wherein the amount of said hearing loss recovery as a result of treatment from the composition is demonstrated by an increased rate of decline in the ITS of a treated mammal as compared to the rate of decline in the TTS of an untreated control mammal, when comparing similar times, 1, 2, or 3 days after sound exposure and the treated mammal shows a greater decrease of at least 9 decibels over the untreated mammal at any of 2, 4, 8, 16, 24 or 32 kHz.

18. A composition of claim 16 for treating hearing loss wherein said composition comprises: at least one scavenger of singlet oxygen, wherein said scavenger is beta-carotene, wherein said beta-carotene scavenger has an activity from about 830 to 5,900 IU, (3 to 21 mg).

19. A composition of claim 18 for treating hearing loss wherein said composition comprises daily therapeutic doses wherein the following active ingredients in the following amounts are in each daily TD dose:
i) vitamin E from 25 to 100 mg;
ii) vitamin C from 50 to 200 mg;
iii) vitamin A from 1 to 8 mg in the form as beta-carotene, and
iiii) magnesium from 25 to 125 mg.

20. A composition of claim 19 for treating hearing loss wherein said composition comprises a combination of all of the active ingredients shown in any columns of Table 2a, wherein the total weight of the capsule in mg is an approximate weight that includes optional components and inactive ingredients used to make the capsule;
TABLE 2

| Component | | Capsule Size | Capsule Size |
|---|---|---|---|
| active ingredient | Capsule Size O | OO | OOO |
| Total weight of | 500-570 | 700-800 | 900-1150 |
| capsule in mg | | | |
| Vitamin E | 34.5-39.3 | 48.3-55.2 | 62.1-79.3 |
| mg active/capsule | | | |
| Vitamin C | 64.3-73.4 | 90.1-103 | 115.8-148 |
| mg active/capsule | | | |
| Vitamin A | 2.3-2.7 | 3.3-3.7 | 4.2-5.3 |
| mg active/capsule | | | |
| as Beta-carotene | | | |
| Magnesium | 40.7-46.4 | 57-65.1 | 73.2-93.6 |

.(period)
